(19) United States
(12) Patent Application Publication
(10) Pub. No.: US 2026/0137732 A1
(43) Pub. Date: May 21, 2026

(54) AKKERMANSIA MUCINIPHILA FOR INCREASING GLUCAGON-LIKE PEPTIDE-1, EXTRACT AND METHOD THEREOF

(71) Applicant: Leeuwenhoek Laboratories Co. Ltd., Taipei City (TW)

(72) Inventors: Chiao-Wei LIN, Taipei City (TW); Yi-Ling LO, Taipei City (TW); Hsin-Bai ZOU, Taipei City (TW); Wen-Hui LIN, Taipei City (TW); Chi-Shan LI, Taipei City (TW); Cheng-Chih HSU, Taipei City (TW)

(21) Appl. No.: 19/392,183

(22) Filed: Nov. 18, 2025

Related U.S. Application Data
(60) Provisional application No. 63/721,573, filed on Nov. 18, 2024.
(60) Provisional application No. 63/738,839, filed on Dec. 26, 2024.

(51) Int. Cl. A61K 35/741; A61K 35/00; C12N 1/205; C12R 1/01

(52) U.S. Cl. CPC A61K 35/741; C12N 1/205; A61K 2035/115; C12R 2001/01

(57) ABSTRACT

Provided is an Akkermansia muciniphila strain including Akkermansia muciniphila LWHK0001, LWHK0002, LWHK0004, LWHK0005, LWHK0006, or LWHK0008. Also provided is a method of Akkermansia muciniphila for increasing glucagon-like peptide-1, including LWHK0001, LWHK0002, LWHK0003, LWHK0004, LWHK0005, LWHK0006, or LWHK0008. Also provided is an Akkermansia muciniphila extract having the effect of increasing glucagon-like peptide-1.

### CROSS-REFERENCE TO RELATED APPLICATION

This application claims priority to U.S. Provisional Application Ser. No. 63/721,573, filed on Nov. 18, 2024, and U.S. Provisional Application Ser. No. 63/738,839, filed on Dec. 26, 2024 and, which are herein incorporated by reference in its entirety.

The Sequence Listing associated with this application is filed in electronic format via EFS-Web and is hereby incorporated by reference into the specification in its entirety. The name of the XML file containing the Sequence Listing is NP-38942-US_SEQ_LIST.xml. The size of the XML file is 20,462,521 bytes, and the XML file was created on Nov. 17, 2025.

### BACKGROUND

### Field of Invention

The present invention relates to Akkermansia muciniphila, extract and method thereof. More particularly, the present invention relates to Akkermansia muciniphila for increasing glucagon-like peptide-1, fractions, extract and method thereof.

### Description of Related Art

Since 1975, the global prevalence of obesity has almost tripled, primarily because of unhealthy eating habits. Since obesity is associated with increased susceptibility to various chronic diseases, the growing prevalence of overweight has become a significant global public health issue. While a considerable number of weight-loss drugs are available, not all of them are effective for obese subjects. Because of the numerous causes of obesity, some obese subjects may have glucagon-like peptide-1 (GLP-1) deficiency.

Therefore, how to increase GLP-1, the related art really needs to be improved.

### SUMMARY

One embodiment of the present disclosure provides an Akkermansia muciniphila selected from the group consisting of: Akkermansia muciniphila LWHK0001 (DSM35271), Akkermansia muciniphila LWHK0002 (DSM35272), Akkermansia muciniphila LWHK0004 (DSM35273), Akkermansia muciniphila LWHK0005 (DSM35274), Akkermansia muciniphila LWHK0006 (DSM35275), and Akkermansia muciniphila LWHK0008 (NITE BP-04475).

Another one embodiment of the present disclosure provides a composition for increasing glucagon-like peptide-1, comprising: the Akkermansia muciniphila as above mentioned; and a pharmaceutical or food-acceptable carrier.

In some embodiments, carrier is selected from micro-crystalline cellulose (MCC), trehalose, maltodextrin, rice flour, magnesium stearate, inositol, dextrose, sucrose, and any combination thereof.

Another one embodiment of the present disclosure provides a method of increasing glucagon-like peptide-1, comprising administering to a subject in need thereof an effective amount of a composition, wherein the composition comprises Akkermansia muciniphila selected from the group consisting of Akkermansia muciniphila LWHK0001, LWHK0002, LWHK0003 (DSM35051), LWHK0004, LWHK0005, LWHK0006, and LWHK0008.

In some embodiments, the Akkermansia muciniphila is live bacteria.

In some embodiments, the composition is a food composition or a pharmaceutical composition.

In some embodiments, composition is administered to a subject orally or parenterally.

Another one embodiment of the present disclosure provides a culture supernatant of Akkermansia muciniphila prepared by the following steps, comprising: providing a bacteria culture medium, wherein the bacteria culture medium comprises the Akkermansia muciniphila selected from the group consisting of LWHK0001, LWHK0002, LWHK0003, LWHK0004, LWHK0005, LWHK0006, and LWHK0008; and centrifuging the bacteria culture medium to obtain a culture supernatant, wherein the culture supernatant comprises a secretion of the Akkermansia muciniphila, a metabolite of the Akkermansia muciniphila, or a combination thereof.

In some embodiments, the Akkermansia muciniphila is the LWHK0008, and the culture supernatant was separated into a fraction with a molecular weight more than 3 kDa, a fraction with a molecular weight from 3 kDa to 10 kDa, a fraction with a molecular weight from 3 kDa to 50 kDa, a fraction with a molecular weight from 50 kDa to 100 kDa, or a fraction with a molecular weight more than 100 kDa.

Another one embodiment of the present disclosure provides a composition with a culture supernatant of Akkermansia muciniphila, comprising: the culture supernatant of Akkermansia muciniphila as above mentioned; and a pharmaceutical or food-acceptable carrier.

Another one embodiment of the present disclosure provides a method of increasing glucagon-like peptide-1, comprising administering to a subject in need thereof an effective amount of a composition, wherein the composition comprises the culture supernatant of Akkermansia muciniphila as above mentioned.

Another one embodiment of the present disclosure provides an extract of Akkermansia muciniphila prepared by the following steps, comprising: providing the culture supernatant of Akkermansia muciniphila as above mentioned, wherein the Akkermansia muciniphila is the LWHK0003; extracting a portion of the culture supernatant with ethyl acetate to obtain an ethyl acetate aqueous layer and an ethyl acetate organic layer; drying the ethyl acetate aqueous layer and the ethyl acetate organic layer respectively, to obtain an ethyl acetate aqueous layer extract and an ethyl acetate organic layer extract respectively; extracting a remaining portion of the culture supernatant with n-hexane to obtain an n-hexane aqueous layer and an n-hexane organic layer; and drying the n-hexane aqueous layer and the n-hexane organic layer respectively, to obtain an n-hexane aqueous layer extract and an n-hexane organic layer extract.

Another one embodiment of the present disclosure provides a composition with extract of Akkermansia muciniphila, comprising: the extract of Akkermansia muciniphila as above mentioned, comprising the ethyl acetate aqueous layer extract, the ethyl acetate organic layer extract, or the n-hexane organic layer extract; and a pharmaceutical or food-acceptable carrier.

Another one embodiment of the present disclosure provides a method of increasing glucagon-like peptide-1, comprising administering to a subject in need thereof an effective amount of a composition, wherein the composition comprises the extract of Akkermansia muciniphila as above mentioned, wherein the extract of Akkermansia muciniphila comprises the ethyl acetate aqueous layer extract, the ethyl acetate organic layer extract, or the n-hexane organic layer extract.

In some embodiments, the composition is food composition or pharmaceutical composition.

In some embodiments, the composition is administered to a subject orally or parenterally.

### BRIEF DESCRIPTION OF THE DRAWINGS

Aspects of the present disclosure are best understood from the following detailed description when read with the accompanying Figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 depicts the differences in blood GLP-1 secretion in mice after glucose ingestion by different AKK strains according to some embodiments of the present disclosure. One-way ANOVA, * indicates p<0.05, ** indicates p<0.01.

FIG. 2 depicts the differences in GLP-1 secretion in NCI-H716 cells after treatment with different AKK culture supernatants according to some embodiments of the present disclosure. T-test, * indicates p<0.05, ** indicates p<0.01.

FIG. 3 depicts the difference in GLP-1 secretion in NCI-H716 cells after treatment with extract of LWHK0003 culture supernatant according to some embodiments of the present disclosure. T-test, * indicates p<0.05, ** indicates p<0.01. Con: an equal volume of dimethyl sulfoxide (DMSO) as the negative control group, CA7S: cholic acid-7-sulfate as the positive control group.

FIG. 4 depicts the differences in GLP-1 secretion in NCI-H716 cells after treatment with LWHK0008 culture supernatant according to some embodiments of the present disclosure. T-test, ** indicates p<0.01.

FIG. 5 depicts the differences in GLP-1 secretion in NCI-H716 cells after treatment with different fractions of LWHK0008 culture supernatant obtained by molecular weight separation according to some embodiments of the present disclosure. T-test, ** indicates p<0.01. BHI: brain heart infusion culture medium.

FIG. 6 depicts the difference in plasma GLP-1 secretion in mice after oral gavage with fraction of LWHK0008 strain with a molecular weight from 3 kDa to 10 kDa according to some embodiments of the present disclosure. T-test, * indicates p<0.05.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the Figures. The spatially relative terms are intended to encompass different orientations of the device in use or operation in addition to the orientation depicted in the Figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number. This repetition is for the purpose of simplicity and clarity and does not in itself dictate a relationship between the various embodiments and/or configurations discussed.

Since there are many causes of obesity, some obese subjects may be patients with GLP-1 deficiency. Supplementing with GLP-1 can slow down the peristalsis of the intestines and reduce the digestion rate, thereby reducing the rate of energy intake and achieving the ideal effect of weight loss.

Some embodiments of the present disclosure provide a Akkermansia muciniphila selected from the group consisting of: Akkermansia muciniphila LWHK0001 (DSM35271), Akkermansia muciniphila LWHK0002 (DSM35272), Akkermansia muciniphila LWHK0004 (DSM35273), Akkermansia muciniphila LWHK0005 (DSM35274), Akkermansia muciniphila LWHK0006 (DSM35275), and Akkermansia muciniphila LWHK0008 (NITE BP-04475).

In some embodiments, the probiotic is live bacteria.

In some embodiments, an amount of the probiotic is from 1×10⁸ CFU/g to 1×10¹¹ CFU/g, for example, 1×10⁸ CFU/g, 2×10⁸ CFU/g, 3×10⁸ CFU/g, 4×10⁸ CFU/g, 5×10⁸ CFU/g, 6×10⁸ CFU/g, 7×10⁸ CFU/g, 8×10⁸ CFU/g, 9×10⁸ CFU/g, 1×10⁹ CFU/g, 2×10⁹ CFU/g, 3×10⁹ CFU/g, 4×10⁹ CFU/g, 5×10⁹ CFU/g, 6×10⁹ CFU/g, 7×10⁹ CFU/g, 8×10⁹ CFU/g, 9×10⁹ CFU/g, 1×10¹⁰ CFU/g, 2×10¹⁰ CFU/g, 3×10¹⁰ CFU/g, 4×10¹⁰ CFU/g, 5×10¹⁰ CFU/g, 6×10¹⁰ CFU/g, 7×10¹⁰ CFU/g, 8×10¹⁰ CFU/g, 9×10¹⁰ CFU/g, 1×10¹¹ CFU/g, or any value between any two of these values.

In some embodiments, the composition is suitable for use in an obese subject. In some examples, the obese subject is caused by following selected from the group consisting of diet, type 2 diabetes, hyperglycemia, glucose intolerance, dyslipidemia, insulin resistance, hyperinsulinemia, fatty liver, cardiovascular disease, stroke, cancer and combinations thereof.

In some embodiments, composition is administered to a subject orally or parenterally. In some embodiments, composition is formulated into an oral dosage form selected from the group consisting of solution, suspension, emulsion, powder, lozenge, pill, syrup, buccal lozenge, tablet, chewing gum and capsule for administration to a subject.

In some embodiments, pharmaceutically acceptable carrier includes, but is not limited to, water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, and other similar or applicable carriers for the present invention.

In some embodiments, composition can be a food composition. For example, edible materials are added in the form of food additives to prepare a food product for human or animal consumption. Food composition includes, but is not limited to, general foods, health foods, beverages, nutritional supplements, dairy products or feeds, etc. In some examples of oral dosage forms, the composition may optionally include pharmaceutical and food-acceptable carriers, excipients and/or additives. In other examples, the dosage form of the composition may include, but is not limited to, powders, tablets, granules, suppositories, microcapsules, ampoules, liquid sprays or suppositories.

In some embodiments, the composition is administered orally or parenterally.

In some embodiments, a culture supernatant of Akkermansia muciniphila was prepared by suspending a certain number of AKK strains in BHI culture medium to form a bacteria culture medium and culturing for 32 hours. In some examples, a certain number of AKK strains include, but are not limited to, from 1×10⁵ CFU/mL to 1×10⁸ CFU/mL, for example, 1×10⁵ CFU/mL, 1×10⁶ CFU/mL, 5×10⁶ CFU/mL, 1×10⁷ CFU/mL, 5×10⁷ CFU/mL, 1×10⁸ CFU/mL, or any value between any two of these values. Next, the bacteria culture medium was centrifuged to obtain a precipitate containing bacteria and a culture supernatant. In some examples, the purpose of centrifugation is to initially isolate and remove the strains while retaining the produced secretions and metabolites during the culture process. Therefore, any known centrifugation speed and time that achieves this effect can be used. Centrifugation conditions include, but are not limited to, 10,000×g for 30 minutes. Next, the culture supernatant may be selectively filtered through a 0.22 μm filter to remove any remaining strains. In some examples, the centrifuged culture supernatant may contain no or a small amount of strains; if a small amount of strains are present, further filtration with a filter membrane can be used to remove any remaining strains.

In some embodiments, an extract of Akkermansia muciniphila is obtained by extracting the portion of the culture supernatant as aforementioned with ethyl acetate (EA) to obtain an ethyl acetate aqueous layer (EA_H₂O) and an ethyl acetate organic layer (EA_EA). In some examples, a volume percentage of the ethyl acetate includes, but is not limited to, 5%, 10%, 20%, 30%, 40%, 50%, 60%, 70%, 80%, 90%, 95%, 99.5%, or any value between any two of these values. The types of substances extracted from the ethyl acetate aqueous layer with different volume percentages of ethyl acetate were roughly the same, and the types of substances extracted from the ethyl acetate organic layer with different volume percentages of ethyl acetate were roughly the same. The only difference was that a higher volume percentage of ethyl acetate resulted in a larger volume of ethyl acetate organic layer, and the more amount of non-polar metabolites can be extracted (with the same types of substances), while a smaller volume of ethyl acetate aqueous layer resulted in less amount of polar metabolites can be extracted (with the same types of substances), and vice versa. The remaining portion of the supernatant was extracted with n-hexane (Hex) to obtain an n-hexane aqueous layer (Hex_H₂O) and an n-hexane organic layer (Hex_Hex). In some examples, a volume percentage of n-hexane includes, but is not limited to, 5%, 10%, 20%, 30%, 40%, 50%, 60%, 70%, 80%, 90%, 95%, 99.5%, or any value between any two of these values. The types of substances extracted from n-hexane aqueous layers with different volume percentages of n-hexane were roughly the same, and the types of substances extracted from the n-hexane organic layers with different volume percentages of n-hexane were roughly the same. The only difference is that a higher volume percentage of n-hexane resulted in a larger volume of n-hexane organic layer, and the more amount of non-polar metabolites can be extracted (with the same types of substances), while a smaller volume of n-hexane aqueous layer resulted in less amount of polar metabolites can be extracted (with the same types of substances), and vice versa.

One embodiment of the present disclosure also provides a use (application) of Akkermansia muciniphila, an extract of Akkermansia muciniphila, a culture supernatant of Akkermansia muciniphila, or a fraction of a culture supernatant of Akkermansia muciniphila for the manufacture of a composition for preventing or treating reduced GLP-1 secretion, wherein the Akkermansia muciniphila is selected from the group consisting of Akkermansia muciniphila LWHK0001, LWHK0002, LWHK0003, LWHK0004, LWHK0005, LWHK0006, and LWHK0008, in which the culture supernatant of Akkermansia muciniphila includes a secretion of the Akkermansia muciniphila, a metabolite of the Akkermansia muciniphila, or a combination thereof, in which the extract of Akkermansia muciniphila includes an ethyl acetate aqueous layer extract, an ethyl acetate organic layer extract, or an n-hexane organic layer extract, in which the culture supernatant of Akkermansia muciniphila includes a fraction with a molecular weight more than 3 kDa, a fraction with a molecular weight from 3 kDa to 10 kDa, a fraction with a molecular weight from 3 kDa to 50 kDa, a fraction with a molecular weight from 50 kDa to 100 kDa, or a fraction with a molecular weight more than 100 kDa.

One embodiment of the present disclosure also provides a use (application) of Akkermansia muciniphila, an extract of Akkermansia muciniphila, a culture supernatant of Akkermansia muciniphila, or a fraction of a culture supernatant of Akkermansia muciniphila for the manufacture of a composition for preventing or treating glucagon-like peptide-1 insufficiency, wherein the Akkermansia muciniphila is selected from the group consisting of Akkermansia muciniphila LWHK0001, LWHK0002, LWHK0003, LWHK0004, LWHK0005, LWHK0006, and LWHK0008, in which the culture supernatant of Akkermansia muciniphila includes a secretion of the Akkermansia muciniphila, a metabolite of the Akkermansia muciniphila, or a combination thereof, wherein the extract of Akkermansia muciniphila includes the ethyl acetate aqueous layer extract, the ethyl acetate organic layer extract, or the n-hexane organic layer extract, wherein the culture supernatant of Akkermansia muciniphila includes a fraction with a molecular weight more than 3 kDa, a fraction with a molecular weight from 3 kDa to 10 kDa, a fraction with a molecular weight from 3 kDa to 50 kDa, a fraction with a molecular weight from 50 kDa to 100 kDa, or a fraction with a molecular weight more than 100 kDa.

One embodiment of the present disclosure also provides a use (application) of Akkermansia muciniphila, an extract of Akkermansia muciniphila, a culture supernatant of Akkermansia muciniphila, or a fraction of a culture supernatant of Akkermansia muciniphila for the manufacture of a composition for preventing or treating glucagon-like peptide-1 deficiency, wherein the Akkermansia muciniphila is selected from the group consisting of Akkermansia muciniphila LWHK0001, LWHK0002, LWHK0003, LWHK0004, LWHK0005, LWHK0006, and LWHK0008, in which the culture supernatant of Akkermansia muciniphila includes a secretion of the Akkermansia muciniphila, a metabolite of the Akkermansia muciniphila, or a combination thereof, in which the extract of Akkermansia muciniphila includes the ethyl acetate aqueous layer extract, the ethyl acetate organic layer extract, or the n-hexane organic layer extract, wherein the culture supernatant of Akkermansia muciniphila includes a fraction with a molecular weight more than 3 kDa, a fraction with a molecular weight from 3 kDa to 10 kDa, a fraction with a molecular weight from 3 kDa to 50 kDa, a fraction with a molecular weight from 50 kDa to 100 kDa, or a fraction with a molecular weight more than 100 kDa.

A number of examples are provided herein to elaborate the composition of the instant disclosure. However, the examples are for demonstration purpose alone, and the instant disclosure is not limited thereto.

A number of examples are provided herein to elaborate the Akkermansia muciniphila, culture supernatant, extract, and use thereof of the instant disclosure for increasing glucagon-like peptide-1. However, the examples are for demonstration purpose alone, and the instant disclosure is not limited thereto.

For the sake of clarity, features and elements that are well known in the art and are not necessary for an understanding of the principles described have been omitted.

### Example 1 Identification of Strains

The strains of Akkermansia muciniphila LWHK0001, 0002, 0003, 0004, 0005, and 0006 of the present disclosure have been deposited at bioresource collection and research center (BCRC) in Taiwan (address of depositary institution: No. 331 Shih-Pin Road, Hsinchu, Taiwan 300), and also have been deposited at Leibniz Institute DSMZ-German Collection of Microorganisms and Cell Cultures GmbH (address of depositary institution: Inhoffenstraβe 7B, 31824 Braunschweig, Germany). The strain of Akkermansia muciniphila LWHK0008 has been deposited at International Patent Organism Depositary (IPOD) of National Institute of Technology and Evaluation (NITE) (address of depositary institution: #122, 2-5-8 Kazusakamatari, Kisarazu-shi, Chiba 292-0818, Japan). Detailed information is shown in Tables 1 and 2 below.

TABLE 1

| | | | Genomic DNA |
|---|---|---|---|
| | | | sequence |
| Strains | Deposit number | Deposit date | SEQ ID NO: |
| LWHK0001 | BCRC911246 | 2024 Nov. 21 | 1 |
| LWHK0002 | BCRC911247 | 2024 Nov. 21 | 2 |
| LWHK0003 | BCRC911209 | 2023 Nov. 21 | 3 |
| LWHK0004 | BCRC911248 | 2024 Nov. 21 | 4 |
| LWHK0005 | BCRC911249 | 2024 Nov. 21 | 5 |
| LWHK0006 | BCRC911250 | 2024 Nov. 21 | 6 |
| LWHK0008 | NITE BP-04475 | 2025 Oct. 22 | 7 |

The strains of Akkermansia muciniphila LWHK0001, 0002, 0003, 0004, 0005, 0006, and 0008 of the present disclosure are extremely anaerobic bacteria, isolated from feces of healthy humans in Taiwan. The strain is oval in shape, with a diameter of approximately from 0.4 to 0.6 micrometers and a length of approximately from 0.6 to 1 micrometer. The Akkermansia muciniphila live bacteria of the present disclosure were cultured in Brain Heart Infusion Broth (BHI) under anaerobic conditions at 37° C. The strains of Akkermansia muciniphila LWHK0001, 0002, 0003, 0004, 0005, 0006, and 0008 have a common sequence as set forth in SEQ ID NO: 8, located from approximately 2234 kbp to 2646 kbp in the LWHK0001 genomic DNA, from approximately 2643 kbp to 2237 kbp in the LWHK0002 genomic DNA, from approximately 603 kbp to 600 kbp in the LWHK0003 genomic DNA, from approximately 658 kbp to 655 kbp in the LWHK0004 genomic DNA, from approximately 2338 kbp to 2341 kbp in the LWHK0005 genomic DNA, from approximately 2318 kbp to 2321 kbp in the LWHK0006 genomic DNA and from approximately 2232 kbp to 2235 kbp in the LWHK0008 genomic DNA, and the common sequence does not present in the standard strain of Akkermansia muciniphila ATCC BAA-835. This common sequence was analyzed using an online system (Open Reading Frame Finding https://www.ncbi.nlm.nih.gov/orffinder/), predicting approximately 20 open reading frames (ORFs). Wherein, ORF 11, with 838 amino acids (SEQ ID NO: 9), did not align to any known genes; ORF 12, with 997 amino acids (SEQ ID NO: 10), should be a gene containing the beta-N-acetylglucosamine (β-NAGase) domain. The β-NAGase is a glycosidase responsible for cleaving the bonds between β-N-acetylglucosamine (β-GlcNAc, β-NAG) and other glycosyl groups. Proteins containing this functional domain are primarily involved in polysaccharide metabolism, protein modification regulation, cell wall degradation, or extracellular matrix (ECM) remodeling.

A whole-genome identification was performed with the strains of Akkermansia muciniphila LWHK0001, 0002, 0003, 0004, 0005, 0006, and 0008 of the present disclosure, and the standard strain ATCC BAA-835 was used as the basis for comparison. The identification items included ANIb (average nucleotide identity on BLAST, calculated based on BLAST+), ANIm (calculated based on MUMmer), Pearson correlation coefficient (PCC, statistical analysis of tetranucleotide usage patterns), and Aligned (aligned to the total nucleotide acids of ATCC BAA-835, 2664102 bp).

TABLE 2

| Genome | ANIb [%] | ANIm [%] | PCC | Aligned [bp] |
|---|---|---|---|---|
| LWHK0001 | 98.56 | 98.90 | 0.99905 | 2483464 |
| LWHK0002 | 87.20 | 88.69 | 0.95334 | 2128605 |
| LWHK0003 | 87.25 | 88.68 | 0.95234 | 2109990 |
| LWHK0004 | 87.12 | 98.84 | 0.95087 | 2102040 |
| LWHK0005 | 98.68 | 88.64 | 0.99877 | 2530209 |
| LWHK0006 | 98.79 | 98.94 | 0.99904 | 2543814 |
| LWHK0008 | 97.22 | 97.51 | 0.99943 | 2450656 |

As shown in Table 2 above, there are differences between the genomes according to the results of the whole genome average nucleotide identity (ANI) analysis with the standard strain ATCC BAA-835, indicating that LWHK0001, LWHK0002, LWHK0003, LWHK0004, LWHK0005, LWHK0006, and LWHK0008 are new isolated strains.

### Example 2 Differences in Blood GLP-1 Secretion Among Different AKK Strains in Mice after Glucose Ingestion

This experiment used 8- to 12-week-old germ-free C57BL/6 male mice, randomly assigned to three experimental groups and one control group. At the start of the experiment, the experimental groups were administered different strains of Akkermansia muciniphila (AKK) via gavage: BAA-835 (residually stored in ATCC and freely available for distribution), LWHK0001, and LWHK0003 live bacteria. At the beginning of the experiment, each group was gavaged with different strains of Akkermansia muciniphila (AKK) once, with a bacterial count of 4×10⁸ CFU, suspended in phosphate-buffered saline (PBS) containing 2.5% glycerol. The control group was gavaged with an equal volume of PBS containing 2.5% glycerol. One week later, mouse feces were collected and prepared into smears for Gram staining to confirm bacterial colonization in the mouse intestines and the absence of other bacterial contamination. Two weeks after gavage administration of live bacteria, a GLP-1 secretion efficacy test was performed.

After mice or humans ingest carbohydrates, fats, and proteins, their intestinal L-cells are induced to secrete GLP-1 into the blood. To compare the differences in the efficacy of different AKK strains in promoting GLP-1 secretion in mice, mice were administrated glucose solution, and the concentration of GLP-1 in the blood of the mice after glucose ingestion was detected. The experimental procedure is as follows: after fasting for 5 hours, mice were given glucose solution via oral gavage at a dose of 2 g/kg BW. Blood was collected 10 minutes later, and dipeptidyl peptidase-4 (DPP4) inhibitor was added to prevent GLP-1 degradation. After separating plasma from the blood sample, the amount of GLP-1 in the sample was analyzed by an ELISA kit.

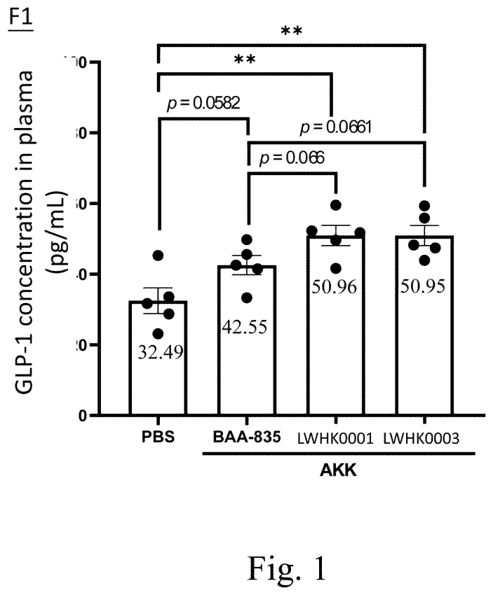

Please refer to drawing F1 of FIG. 1. After different AKK strains colonized in mice for 2 weeks, the differences in blood GLP-1 secretion in different groups of mice after glucose ingestion were tested. The blood GLP-1 concentration in the control group was 32.49 pg/mL, the blood GLP-1 concentration in the BAA-835 group was 42.55 pg/mL, the blood GLP-1 concentration in the LWHK0001 group was 50.96 pg/mL, and the blood GLP-1 concentration in the LWHK0003 group was 50.95 pg/mL. Statistical analysis showed that the blood GLP-1 levels in mice colonized by LWHK0001 and LWHK0003 strains were significantly higher than those in the control group and the BAA-835 group. The above experimental results show that the AKK BAA-835, LWHK0001, and LWHK0003 live bacteria all have the effect of promoting GLP-1 secretion in mice, wherein the AKK LWHK0001 and LWHK0003 are more effective than the AKK BAA-835.

### Example 3 Differences in GLP-1 Secretion in NCI-H716 Cells after Treatment with Different AKK Culture Supernatants

A preparation method of AKK culture supernatant: A suspension of 1×10⁶ CFU/mL AKK strain was cultured in BHI medium for 32 hours. Next, the culture medium was centrifuged at 10,000×g, 4° C., for 30 minutes to obtain a precipitate containing bacteria and a culture supernatant. Next, the culture supernatant was then filtered through a 0.22 μm filter to remove any remaining bacteria. Finally, the culture supernatant was concentrated 5-fold using a centrifuge tube. This 5-fold concentrated AKK culture supernatant was used for subsequent cell experiments.

Cell experiment procedure: The NCI-H716 cell line was used as a cell model (this cell line was obtained from colorectal adenocarcinoma ascites and is currently used as a human model for in vitro research on GLP-1 regulation). The NCI-H716 cell line was cultured in RPMI 1640 medium containing 10% fetal bovine serum (FBS). Next, the cells were seeded on a matrix-coated 96-well plate (Matrigel®) at a cell number of 6×10⁴ cells/well. Next, after culturing the cells on the matrix-coated 96-well plate for 2 days, the experiment was performed. Next, the cell culture medium was removed, and the cells were washed once with Hank's balanced salt solution (HBSS). Next, the HBSS was removed, and HBSS containing 180 μL of 0.2% BSA was added to the cells and treated for 2 hours. Next, 20 μL of 5-fold concentrated AKK culture supernatant was added into the experimental group, while the control group was added with the same volume of HBSS for 2 hours. Next, cell culture medium was collected and Diprotin A (final concentration 10 μM) was added (Note: Diprotin A is a DPP4 inhibitor, inhibiting the DPP4 enzyme to prevent GLP-1 from being broken down by DPP4). Finally, the GLP-1 concentration in the cell culture medium of different groups was analyzed by an ELISA kit.

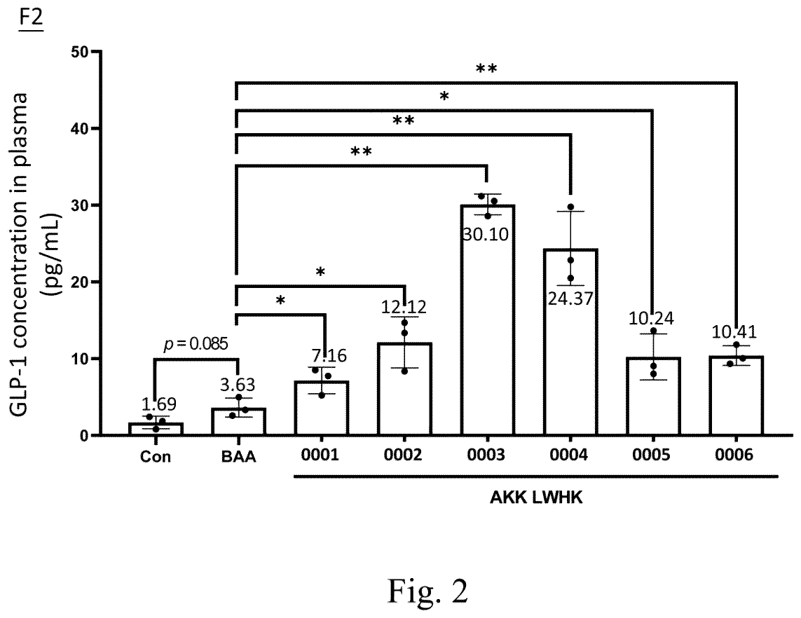

Please refer to drawing F2 of FIG. 2, FIG. 2 shows that after treating different groups of NCI-H716 cells with culture supernatants of AKK BAA-835, LWHK0001, LWHK0002, LWHK0003, LWHK0004, LWHK0005, and LWHK0006 or HBSS for 2 hours, the GLP-1 concentration was 1.69 pg/mL in the control group, 3.63 pg/mL in the BAA-835 group, 7.16 pg/mL in the LWHK0001 group, 12.12 pg/mL in the LWHK0002 group, 30.10 pg/mL in the LWHK0003 group, 24.37 pg/mL in the LWHK0004 group, 10.24 pg/mL in the LWHK0005 group, and 10.41 pg/mL in the LWHK0006 group. Statistical analysis showed that the culture supernatants of the AKK LWHK0001, LWHK0002, LWHK0003, LWHK0004, LWHK0005, and LWHK0006 were significantly more effective than the culture supernatant of the AKK BAA-835 in inducing GLP-1 secretion in NCI-H716 cells. These results indicate that the culture supernatants of the AKK BAA-835, LWHK0001, LWHK0002, LWHK0003, LWHK0004, LWHK0005, and LWHK0006 all promoted GLP-1 secretion, and the AKK LWHK0001, LWHK0002, LWHK0003, LWHK0004, LWHK0005, and LWHK0006 were more effective than the AKK BAA-835.

### Example 4 Differences in GLP-1 Secretion after Treatment of Different Portions of Extract of LWHK0003 Culture Supernatant

A preparation method of AKK extract of LWHK0003 culture supernatant: A suspension of 1×10⁶ CFU/mL AKK strain was cultured in BHI medium for 32 hours. Next, the culture medium was centrifuged at 10,000×g, 4° C., for 30 minutes to obtain a precipitate containing bacteria and a culture supernatant. Next, the culture supernatant was then filtered through a 0.22 μm filter to remove any remaining bacteria. Next, a portion of culture supernatant was extracted with 99.5% ethyl acetate (EA) to obtain an ethyl acetate aqueous layer (EA_H₂O) and an ethyl acetate organic layer (EA_EA); a remaining portion of the culture supernatant was extracted with 95% n-hexane (Hex) to obtain an n-hexane aqueous layer (Hex_H₂O) and an n-hexane organic layer (Hex_Hex). Next, the ethyl acetate aqueous layer (EA_H₂O) and the ethyl acetate organic layer (EA_EA), and the n-hexane aqueous layer (Hex_H₂O) and the n-hexane organic layer (Hex_Hex) were dried, respectively, to obtain an ethyl acetate aqueous layer extract and an ethyl acetate organic layer extract, an n-hexane aqueous layer extract and an n-hexane organic layer extract. Uncultured BHI culture medium was also extracted and dried using the method described above. The dried 20 mg sample was redissolved in 0.5 mL of DMSO and used for cell experiments.

Cell experiment procedure: The NCI-H716 cell line was used as a cell model. The NCI-H716 cell line was cultured in RPMI 1640 medium containing 10% FBS. Next, the cells were seeded on a matrix-coated 96-well plate at a cell number of 6×10⁴ cells/well. Next, after culturing the cells on the matrix-coated 96-well plate for 2 days, the experiment was performed. Next, the cell culture medium was removed, and the cells were washed once with HBSS. Next, the HBSS was removed, and HBSS containing 200 μL of 0.2% BSA was added to the cells and treated for 2 hours. Next, the HBSS containing 0.2% BSA was removed. Next, HBSS containing 0.2% BSA and the extract was added; the control group was treated with HBSS containing 0.2% BSA and DMSO as the same volume as the extract and treated for 2 hours. Next, a cell culture medium was collected and Diprotin A (final concentration 10 μM) was added. Next, the collected cell culture medium was used to analyze the GLP-1 concentration in the cell culture medium of different groups using an ELISA kit.

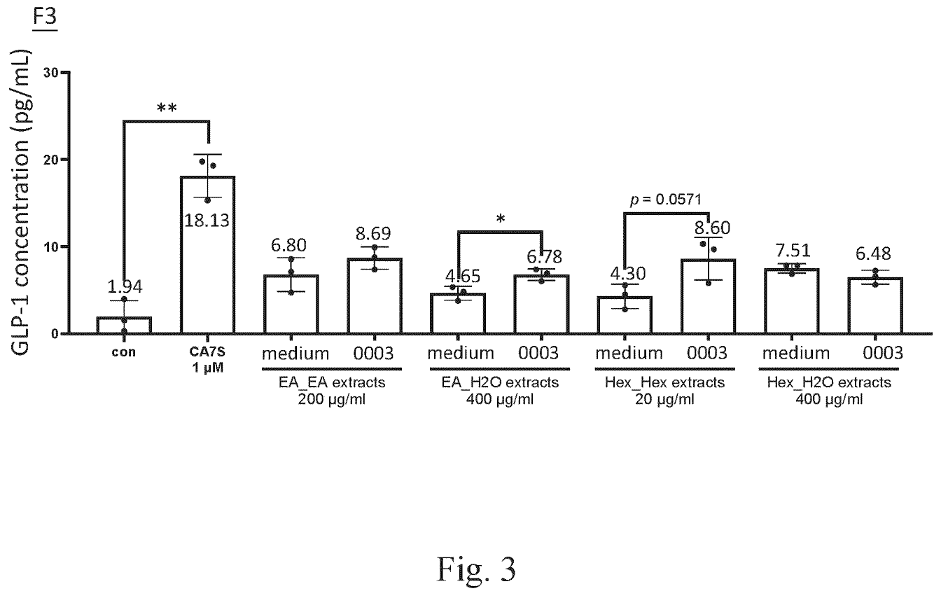

Please refer to drawing F3 of FIG. 3, FIG. 3 shows that after treating different groups of NCI-H716 cells with different extracts of the AKK LWHK0003 culture supernatant for 2 hours, the GLP-1 concentration was 1.94 pg/mL in the control group, 18.13 pg/mL in 1 μM CA7S, 6.80 pg/mL in the EA_EA extract of uncultured BHI culture medium, 8.69 pg/mL in the EA_EA extract of the AKK LWHK0003 culture supernatant, 4.65 pg/mL in the EA_H₂O extract of uncultured BHI culture medium, 6.78 pg/mL in the EA_H₂O extract of the AKK LWHK0003 culture supernatant, 4.30 pg/mL in the Hex_Hex extract of uncultured BHI culture medium, 8.60 pg/mL in the Hex-Hex extract of the AKK LWHK0003 culture supernatant, 7.51 pg/mL in the Hex_H₂O extract of uncultured BHI culture medium, and 6.48 pg/mL in the Hex-H₂O extract of the AKK LWHK0003 culture supernatant. Statistical analysis shows that the EA-H₂O extract of the AKK LWHK0003 culture supernatant was significantly more effective than the EA-H₂O extract from the uncultured BHI culture medium in inducing GLP-1 secretion in NCI-H716 cells; the Hex-Hex extract of the AKK LWHK0003 culture supernatant was significantly more effective than the Hex-Hex extract of the uncultured BHI culture medium in inducing GLP-1 secretion in NCI-H716 cells.

### Example 5

A preparation method of AKK LWHK0008 culture supernatant: The preparation method is the same as in Example 3, except that the strain used here is AKK LWHK0008. This 5-fold concentrated AKK LWHK0008 culture supernatant was used in the experimental group. The uncultured BHI culture medium was concentrated 5 times by a centrifuge tube and used as the control group.

Cell experiment procedure: The NCI-H716 cell line was used as a cell model, and the NCI-H716 cell line was cultured in RPMI 1640 medium containing 10% FBS. Next, the cells were seeded on a matrix-coated 96-well plate at a cell number of 6×10⁴ cells/well. Next, after culturing the cells on the matrix-coated 96-well plate for 2 days, the experiment was performed. Next, the cell culture medium was removed, and the cells were washed once with HBSS. Next, the HBSS was removed, and HBSS containing 180 μL of 0.2% BSA was added to the cells and treated for 2 hours. Next, 20 μL of 5-fold concentrated AKK LWHK0008 culture supernatant was added into the experimental group, while the control group was added with the same volume of 5-fold concentrated BHI culture medium containing uncultured bacteria for 2 hours. Next, cell culture medium was collected and Diprotin A (final concentration 10 μM) was added (Note: Diprotin A is a DPP4 inhibitor, inhibiting the DPP4 enzyme to prevent GLP-1 from being broken down by DPP4). Finally, the collected cell culture media were analyzed using an ELISA kit to determine the GLP-1 concentration in the cell culture media of different groups.

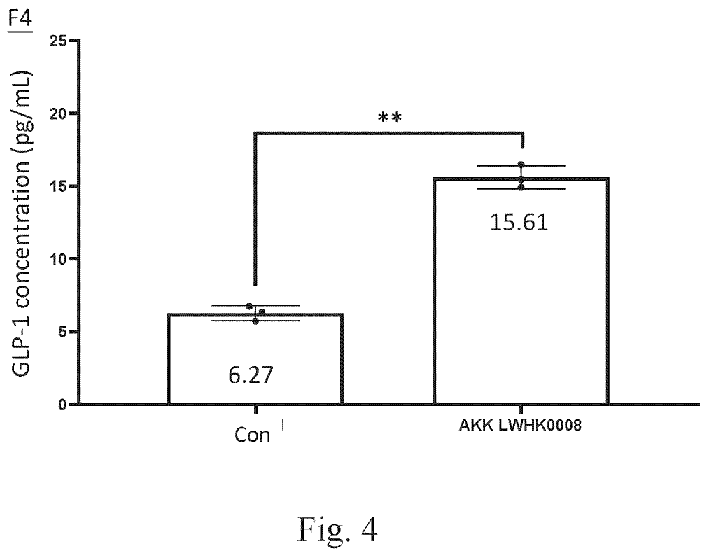

Please refer to drawing F4 of FIG. 4, after treating different groups of NCI-H716 cells with either AKK LWHK0008 culture supernatant or uncultured BHI medium for 2 hours, the GLP-1 concentration in the control group was 6.27 pg/mL, while the GLP-1 concentration in the LWHK0008 group was 15.61 pg/mL. Statistical analysis shows that the LWHK0008 culture medium was significantly more effective than the control group in inducing GLP-1 secretion in NCI-H716 cells. These results indicate that AKK LWHK0008, after culturing, produces a product that promotes GLP-1 secretion in the culture medium.

### Example 6

A preparation method of AKK LWHK0008 culture supernatant with different molecular weight fractions: AKK LWHK0008 at a concentration of 1×10⁶ CFU/mL cultured in BHI medium became a bacteria culture medium for 32 hours. Next, the bacteria culture medium was centrifuged at 10,000×g, 4° C., for 30 minutes to obtain a precipitate containing bacteria and a culture supernatant. Next, the culture supernatant was then filtered through a 0.22 μm filter to remove any remaining bacteria. Next, the filtered culture supernatant was added into a 3 kDa molecular weight cut-off (MWCO) centrifuge tube and centrifuged to obtain concentrates <3 kDa and >3 kDa. Next, a portion of the >3 kDa concentrate was added into a 50 kDa MWCO centrifuge tube and centrifuged to obtain concentrates of 3-50 kDa and >50 kDa. Next, the >50 kDa concentrate was added into a 100 kDa MWCO centrifuge tube and centrifuged to obtain concentrates of 50-100 kDa and >100 kDa. Finally, the concentrates of >3 kDa, 3-50 kDa, 50-100 kDa, and >100 kDa were used for subsequent cell experiments. Uncultured BHI culture medium was also separated using the above method and used as a control sample.

Cell experiment procedure: The NCI-H716 cell line was used as a cell model, and the NCI-H716 cell line was cultured in RPMI 1640 medium containing 10% FBS. Next, the cells were seeded on a matrix-coated 96-well plate at a cell number of 6×10⁴ cells/well. Next, after culturing the cells on the matrix-coated 96-well plate for 2 days, the experiment was performed. Next, the cell culture medium was removed, and the cells were washed once with HBSS. Next, the HBSS was removed, and HBSS containing 180 μL of 0.2% BSA was added to the cells and treated for 2 hours. Next, 20 μL of culture supernatant fractions were added to the experimental group, while the control group was added with the same volume of uncultured BHI culture fractions for 2 hours. Next, cell culture medium was collected and Diprotin A (final concentration 10 μM) was added (Note: Diprotin A is a DPP4 inhibitor, inhibiting the DPP4 enzyme to prevent GLP-1 from being broken down by DPP4). Finally, the collected cell culture media were analyzed using an ELISA kit to determine the GLP-1 concentration in the cell culture media of different groups.

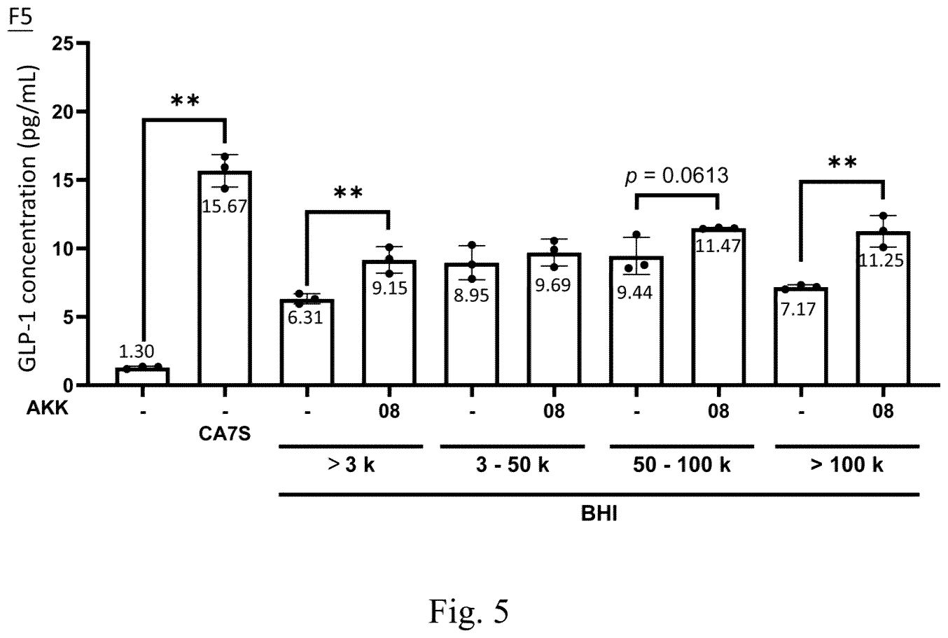

Please refer to drawing F5 of FIG. 5, fractions of AKK LWHK0008 culture medium or uncultured BHI culture medium were used to treat different groups of NCI-H716 cells for 2 hours. In the fraction >3 kDa, the GLP-1 concentration in the control group was 6.31 pg/mL, and the GLP-1 concentration in the LWHK0008 group was 9.15 pg/mL. In the fractions 3-50 kDa, the GLP-1 concentration in the control group was 8.95 pg/mL, and the GLP-1 concentration in the LWHK0008 group was 9.69 pg/mL; in the fraction 50-100 kDa, the GLP-1 concentration in the control group was 9.44 pg/mL, and the GLP-1 concentration in the LWHK0008 group was 11.47 pg/mL; in the fraction >100 kDa, the GLP-1 concentration in the control group was 7.17 pg/mL, the GLP-1 concentration in the LWHK0008 group was 11.25 pg/mL. Statistical analysis shows that the fractions >3 kDa and >100 kDa of LWHK0008 culture supernatant were significantly more effective than the control group in inducing GLP-1 secretion in NCI-H716 cells, while the fraction 50-100 kDa of LWHK0008 culture supernatant showed a trend towards higher than the control group in inducing GLP-1 secretion in NCI-H716 cells. These results indicate that AKK LWHK0008, after cultivation, produces a product that promotes GLP-1 secretion in the culture medium. The molecular weight range of the active substances >3 kDa, 3-50 kDa, 50-100 kDa, and >100 kDa produce a product that promotes GLP-1 secretion in the culture medium.

### Example 7

A preparation method of a fraction 3-10 kDa of AKK LWHK0008 culture supernatant: AKK LWHK0008 at a concentration of 1×10⁶ CFU/mL cultured in BHI medium became a bacteria culture medium for 32 hours. Next, the bacteria culture medium was centrifuged at 10,000×g, 4° C., for 30 minutes to obtain a precipitate containing bacteria and a culture supernatant. Next, the culture supernatant was then filtered through a 0.22 μm filter to remove any remaining bacteria. Next, the filtered culture supernatant was added into a 3 kDa molecular weight cut-off (MWCO) centrifuge tube and centrifuged to obtain concentrates <3 kDa and >3 kDa. Next, the >3 kDa concentrate was added into a 10 kDa MWCO centrifuge tube and centrifuged to obtain concentrates of 3-10 kDa and >10 kDa. Finally, the concentrates of 3-10 kDa was used for subsequent animal experiments. Uncultured BHI culture medium was also separated using the above method and used as a control sample.

Cell experiment procedure: Eight-week-old male C57BL/6 mice were randomly assigned to an experimental group and a control group, with eight mice in each group. On the day of the experiment, the mice were fasted for 4 hours, and then each mouse was given 200 μL of sample via oral gavage. Five hours after tube feeding, blood was collected and a DPP4 inhibitor was added to prevent GLP-1 degradation. After separating plasma from the blood samples, the GLP-1 content in the samples was analyzed using an ELISA kit.

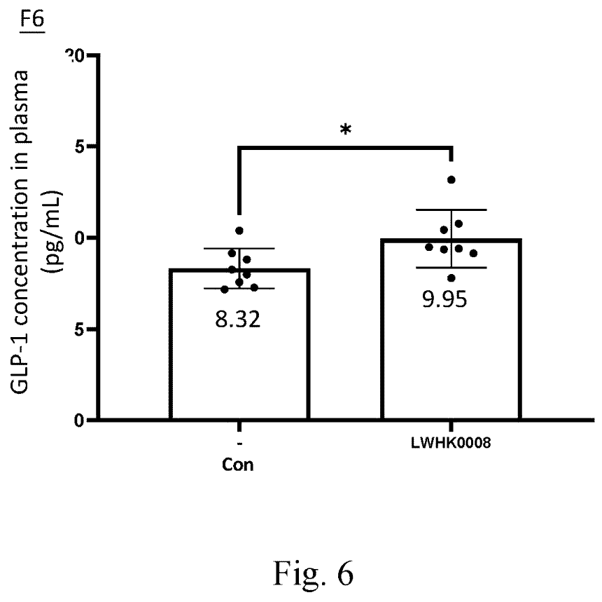

Please refer to drawing F6 in FIG. 6. Mice that had been fasted for 4 hours were given BHI culture medium without culture and the fraction 3-10 kDa of LWHK0008 culture supernatant for 5 hours. The blood GLP-1 concentration in the control group was 8.32 pg/mL, while the concentration in the LWHK0008 group was 9.95 pg/mL. Statistical analysis shows that the blood GLP-1 concentration in the LWHK0008 group was significantly higher than that in the control group. These results indicate that the fraction 3-10 kDa of LWHK0008 culture supernatant can promote GLP-1 secretion.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An Akkermansia muciniphila selected from the group consisting of:
Akkermansia muciniphila LWHK0001 (DSM35271),
Akkermansia muciniphila LWHK0002 (DSM35272),
Akkermansia muciniphila LWHK0004 (DSM35273),
Akkermansia muciniphila LWHK0005 (DSM35274),
Akkermansia muciniphila LWHK0006 (DSM35275), and
Akkermansia muciniphila LWHK0008 (NITE BP-04475).

2. A composition for increasing glucagon-like peptide-1, comprising:
the Akkermansia muciniphila as claimed in claim 1; and
a pharmaceutical or food-acceptable carrier.

3. The composition of claim 2, wherein the carrier is selected from micro-crystalline cellulose, trehalose, maltodextrin, rice flour, magnesium stearate, inositol, dextrose, sucrose, and any combination thereof.

4. A method of increasing glucagon-like peptide-1, comprising administering to a subject in need thereof an effective amount of a composition, wherein the composition comprises Akkermansia muciniphila selected from the group consisting of Akkermansia muciniphila LWHK0001, LWHK0002, LWHK0003 (DSM35051), LWHK0004, LWHK0005, LWHK0006, and LWHK0008.

5. The method of claim 4, wherein the Akkermansia muciniphila is live bacteria.

6. The method of claim 4, wherein the composition is a food composition or a pharmaceutical composition.

7. The method of claim 4, wherein the composition is administered to a subject orally or parenterally.

8. A culture supernatant of Akkermansia muciniphila prepared by the following steps, comprising:
providing a bacteria culture medium, wherein the bacteria culture medium comprises the Akkermansia muciniphila selected from the group consisting of LWHK0001, LWHK0002, LWHK0003, LWHK0004, LWHK0005, LWHK0006, and LWHK0008; and
centrifuging the bacteria culture medium to obtain a culture supernatant, wherein the culture supernatant comprises a secretion of the Akkermansia muciniphila, a metabolite of the Akkermansia muciniphila, or a combination thereof.

9. The culture supernatant of Akkermansia muciniphila of claim 8, wherein the Akkermansia muciniphila is the LWHK0008, and the culture supernatant was separated into a fraction with a molecular weight more than 3 kDa, a fraction with a molecular weight from 3 kDa to 10 kDa, a fraction with a molecular weight from 3 kDa to 50 kDa, a fraction with a molecular weight from 50 kDa to 100 kDa, or a fraction with a molecular weight more than 100 kDa.

10. A composition with a culture supernatant of Akkermansia muciniphila, comprising:
the culture supernatant of Akkermansia muciniphila as claimed in claim 8; and
a pharmaceutical or food-acceptable carrier.

11. A composition with a culture supernatant of Akkermansia muciniphila, comprising:
the culture supernatant of Akkermansia muciniphila as claimed in claim 9; and
a pharmaceutical or food-acceptable carrier.

12. A method of increasing glucagon-like peptide-1, comprising administering to a subject in need thereof an effective amount of a composition, wherein the composition comprises the culture supernatant of Akkermansia muciniphila as claimed in claim 8.

13. A method of increasing glucagon-like peptide-1, comprising administering to a subject in need thereof an effective amount of a composition, wherein the composition comprising the culture supernatant of Akkermansia muciniphila as claimed in claim 9.

14. An extract of Akkermansia muciniphila prepared by the following steps, comprising:
providing the culture supernatant of Akkermansia muciniphila as claimed in claim 8, wherein the Akkermansia muciniphila is the LWHK0003;
extracting a portion of the culture supernatant with ethyl acetate to obtain an ethyl acetate aqueous layer and an ethyl acetate organic layer;
drying the ethyl acetate aqueous layer and the ethyl acetate organic layer respectively, to obtain an ethyl acetate aqueous layer extract and an ethyl acetate organic layer extract respectively;
extracting a remaining portion of the culture supernatant with n-hexane to obtain an n-hexane aqueous layer and an n-hexane organic layer; and
drying the n-hexane aqueous layer and the n-hexane organic layer respectively, to obtain an n-hexane aqueous layer extract and an n-hexane organic layer extract.

15. A composition with extract of Akkermansia muciniphila, comprising:
the extract of Akkermansia muciniphila as claimed in claim 14, comprising the ethyl acetate aqueous layer extract, the ethyl acetate organic layer extract, or the n-hexane organic layer extract; and
a pharmaceutical or food-acceptable carrier.

16. A method of increasing glucagon-like peptide-1, comprising administering to a subject in need thereof an effective amount of a composition, wherein the composition comprises the extract of Akkermansia muciniphila as claimed in claim 14, wherein the extract of Akkermansia muciniphila comprises the ethyl acetate aqueous layer extract, the ethyl acetate organic layer extract, or the n-hexane organic layer extract.

17. The method of claim 16, wherein the composition is a food composition or a pharmaceutical composition.

18. The method of claim 16, wherein the composition is administered to a subject orally or parenterally.
